(19) United States
(12) Patent Application Publication
(10) Pub. No.: US 2012/0251447 A1
(43) Pub. Date: Oct. 4, 2012

(54) RADIOIODINATED TROPANE DERIVATIVES

(76) Inventors: Michelle Emma Avory, Amersham (GB); Harry John Wadsworth, Amersham (GB); Robert James Domett Nairne, Amersham (GB)

(21) Appl. No.: 13/513,650

(22) PCT Filed: Dec. 21, 2010
(86) PCT No.: PCT/EP2010/070352

§ 371 Date: Jun. 4, 2012

(30) Foreign Application Priority Data
Dec. 22, 2009 (GB) 0922304.1

(51) Int. Cl. C07D 451/02; A61K 51/04

(52) U.S. Cl. 424/1.85; 546/125

(57) ABSTRACT

The present invention provides novel radio iodinated tropanes incorporating triazole or isoxazole rings. Also provided are methods of preparation of said tropanes from functionalised tropane precursors, using click cycloaddition chemistry, as well as radiopharmaceutical compositions comprising such radio iodinated tropanes. The invention also provides in vivo imaging methods using the radio iodinated tropanes.

### FIELD OF THE INVENTION

The present invention provides novel radioiodinated tropanes. Also provided are methods of preparation of said tropanes from functionalised tropane precursors, using click cycloaddition chemistry, as well as radiopharmaceutical compositions comprising such radioiodinated tropanes. The invention also provides in vivo imaging methods using the radioiodinated tropanes.

### BACKGROUND TO THE INVENTION

Radiopharmaceutical imaging agents derived from tropanes are known, and include ¹²³I-CIT (Dopascan™), ¹²³I-CIT-FP (DaTSCAN™) and the E isomer of ¹²³I-2β-carbomethoxy-3 β-(4-fluorophenyl)-N-(1-iodoprop-1-en-3-yl)nortropane (Altropane™). These and other tropane-based imaging agents are described by Morgan and Nowotnik [Drug News Perspect., 12(3), 137-145 (1999):

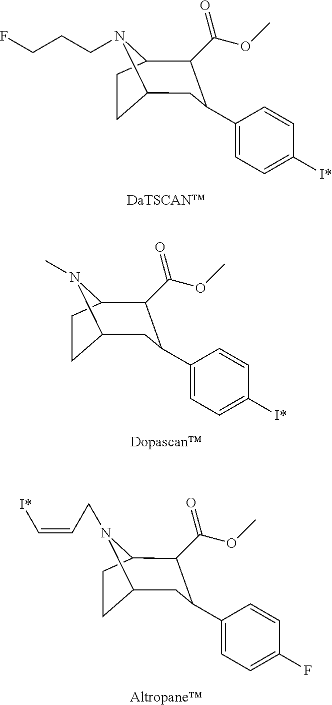

where I* is the radioactive iodine isotope ¹²³I. The agents are useful for imaging the dopamine transporter in vivo, and in particular Parkinsonian syndromes, including Parkinson's disease; DLB (Lewy Body Dementia) and AD-HD.

The applications of “click chemistry” in biomedical research, including radiochemistry, have been reviewed by Nwe et al [Cancer Biother. Radiopharm., 24(3), 289-302 (2009)]. As noted therein, the main interest has been in the PET radioisotope ¹⁸F (and to a lesser extent ¹¹C), plus “click to chelate” approaches for radiometals suitable for SPECT imaging such as ^{99m}Tc or ¹¹¹In. ¹⁸F click-labelling of targeting peptides, giving products incorporating an ¹⁸F-fluoroalkyl-substituted triazole have been reported by Li et al [Bioconj. Chem., 18(6), 1987-1994 (2007)], and Hausner et al [J. Med. Chem., 51(19), 5901-5904 (2008)]. WO 2006/067376 discloses a method for labelling a vector comprising reaction of a compound of formula (I) with a compound of formula (II):

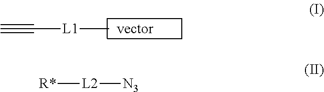

or, a compound of formula (III) with a compound of formula (IV):

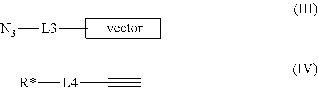

in the presence of a Cu(I) catalyst, wherein:

L1, L2, L3, and L4 are each Linker groups;

R* is a reporter moiety which comprises a radionuclide;

to give a conjugate of formula (V) or (VI) respectively:

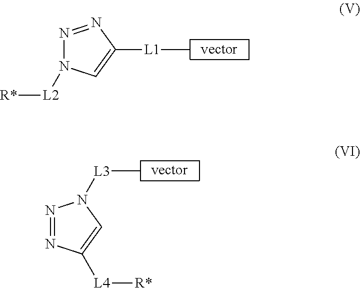

R* of WO 2006/067376 is a reporter moiety which comprises a radionuclide, e.g. a positron-emitting radionuclide. Suitable positron-emitting radionuclides for this purpose are said to include ¹¹C, ¹⁸F, ⁷⁵Br, ⁷⁶Br, ¹²⁴I, ⁸²Rb, ⁶⁸Ga, ⁶⁴Cu and ⁶²Cu, of which ¹¹C and ¹⁸F are preferred. Other useful radionuclides are stated to include ¹²³I, ¹²⁵I, ¹³¹I, ²¹¹At, ^{99m}Tc, and ¹¹¹In.

WO 2007/148089 discloses a method for radiolabelling a vector comprising reaction of a compound of formula (I) with a compound of formula (II):

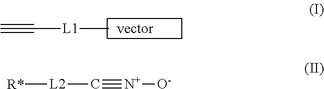

or, a compound of formula (III) with a compound of formula (IV):

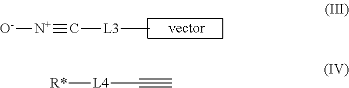

in the presence of a Cu(I) catalyst, wherein:

L1, L2, L3, and L4 are each Linker groups;

R* is a reporter moiety which comprises a radionuclide;

to give a conjugate of formula (V) or (VI) respectively:

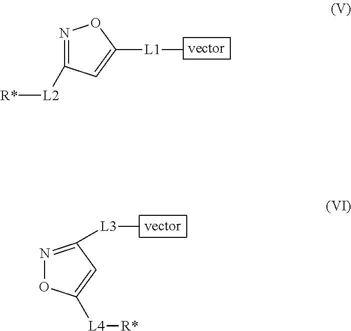

In both WO 2006/067376 and WO 2007/148089, metallic radionuclides are stated to be suitably incorporated into a chelating agent, for example by direct incorporation by methods known to the person skilled in the art. Neither WO 2006/067376 nor WO 2007/148089 discloses any methodology specific for click radioiodination—in particular which combination of compounds of formulae (I)-(IV), together with which combinations of linker groups L1, L2, L3, L4, and which type of R* group would be suitable. In addition, WO 2006/067376 focuses on ¹⁸F, and fluoroacetylene would not be an attractive intermediate for radiolabelling, since it boils at −80° C. and is reported to be explosively unstable in the liquid state [Middleton, J. Am. Chem. Soc., 81, 803-804 (1959)].

There is still a need for alternative radioiodinated tropanes with the potential to image the dopamine transporter in vivo.

The Present Invention.

The present invention provides radioiodinated tropanes which comprise triazole and isoxazole rings. The triazole and isoxazole rings do not hydrolyse and are highly stable to oxidation and reduction, meaning that the labelled tropane has high in vivo stability. The triazole ring is also comparable to an amide in size and polarity. The triazole and isoxazole rings of the products of Formula (I) of the present invention are not, however, expected to be recognized by thyroid deiodination enzymes known to metabolise iodo-tyrosine more rapidly than iodobenzene, and are thus expected to be sufficiently stable in vivo for radiopharmaceutical imaging and/or radiotherapy.

The radioiodinated tropanes of the present invention are useful for imaging the dopamine transporter in vivo. The compounds of the present invention have the radioiodine directly bonded to a triazole or isoxazole heteroaryl ring. The radioiodinated products are thus expected to exhibit good stability with respect to metabolic deiodination in vivo, with consequent unwanted stomach and/or thyroid uptake of radioiodine. The products are therefore suitable for use as radiopharmaceuticals for in vivo imaging, which is an important advantage.

The compounds of Formula (I) may also be conveniently prepared via click radioiodination methodology, which is also readily adaptable to use with an automated synthesizer apparatus. In that regard, the volatility of the iodoacetylene (H-≡-I) used, 32° C. at ca. 1 atmosphere pressure, can be used advantageously to permit facile distillation of the reactive radioiodine species prior to radiolabelling, so that the radiochemical purity (RCP) of the product is maximised. That minimises the need for further product purification processes, such as via chromatography. It is also in contrast with conventional radioiodination methodology, where volatile radioiodine-containing species (e.g. molecular iodine I₂) would be regarded as undesirable due to the increased risks of loss of radioactivity and/or radiation dose.

The compounds of Formula (I) may also be conveniently prepared from organometallic precursors under mild conditions, which avoid the need to manipulate iodoacetylene.

### DETAILED DESCRIPTION OF THE INVENTION

In a first aspect, the present invention provides a radioiodinated tropane of Formula (I):

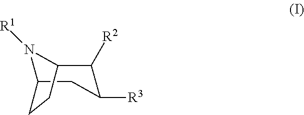

where:
R¹ is C₁₋₄ alkyl, C₁₋₄ fluoro alkyl or Y;
R² is —CO₂R or Y, R is C₁₋₄ alkyl, C₅₋₈ aryl or C₅₋₁₀ aralkyl;
R³ is Y or R⁴, where R⁴ is of formula:

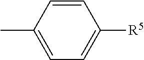

where R⁵ is Hal, CH₃ or Y;
Y is a Y¹ or Y² group:

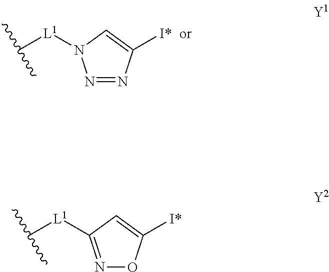

L¹ is a linker group which may be present or absent;
I* is a radioisotope of iodine;
wherein R¹ to R⁵ are chosen such that the tropane of Formula (I) comprises one Y group.

The term “radioiodinated” has its conventional meaning, i.e. a radiolabelled compound wherein the radioisotope used for the radiolabelling is a radioisotope of iodine. The term “radioisotope of iodine” has its conventional meaning, i.e. an isotope of the element iodine that is radioactive. Suitable such radioisotopes include: ¹²³I, ¹²⁴I, ₁₂₅I and ¹³¹I.

The term “tropane” also has its conventional meaning in the field or organic chemistry, and refers to the unsubstituted bicyclic amine of Formula I, i.e. without the substituents R¹, R² and R³.

By the term “linker group” is meant a bivalent group comprising a chain of covalently-bonded atoms which joins two other moieties together via covalent bonds. Preferably, the linker group is unbranched. Preferred linker groups are described below.

Preferred Aspects.

A preferred tropane of the first aspect is where Y is Y¹, i.e. the radioiodine isotope is attached to a triazole ring. Preferred radioisotopes of iodine for use in the present invention are those suitable for medical imaging in vivo using PET or SPECT, preferably ¹²³I, ¹²⁴I or ¹³¹I, more preferably ¹²³I or ¹²⁴I, most preferably ¹²³I.

The tropane may be of synthetic or natural origin, but is preferably synthetic. The term “synthetic” has its conventional meaning, i.e. man-made as opposed to being isolated from natural sources eg. from the mammalian body. Such compounds have the advantage that their manufacture and impurity profile can be fully controlled.

In Formula (I), preferred linker groups (L¹ ) are synthetic, and comprise a group of formula -(A)ₘ- wherein each A is independently —CR₂—, 13 CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block; wherein each R is independently chosen from: H, C₁₋₄ alkyl, C₂₋₄ alkenyl, C₂₋₄ alkynyl, C₁₋₄ alkoxyalkyl or C₁₋₄ hydroxyalkyl; and m is an integer of value 1 to 20.

By the term “peptide” is meant a compound comprising two or more amino acids, as defined below, linked by a peptide bond (ie. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also “Synthesis of Peptides and Peptidomimetics”, M. Goodman et al, Houben-Weyl E22c, Thieme.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

When L¹ comprises a peptide chain of 1 to 10 amino acid residues, the amino acid residues are preferably chosen from glycine, lysine, arginine, aspartic acid, glutamic acid or serine. When L¹ comprises a PEG moiety, it preferably comprises units derived from oligomerisation of the monodisperse PEG-like structures of Formulae Bio1 or Bio2:

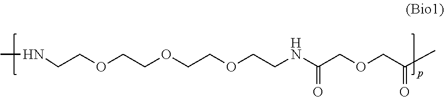

17-amino-5-oxo-6-aza-3, 9, 12, 15-tetraoxaheptadecanoic acid of Formula Bio1 wherein p is an integer from 1 to 10. Alternatively, a PEG-like structure based on a propionic acid derivative of Formula Bio2 can be used:

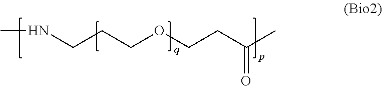

where p is as defined for Formula Bio1 and q is an integer from 3 to 15. In Formula Bio2, p is preferably 1 or 2, and q is preferably 5 to 12.

When the linker group does not comprise PEG or a peptide chain, preferred L¹ groups have a backbone chain of linked atoms which make up the -(A)ₘ- moiety of 2 to 10 atoms, most preferably 2 to 5 atoms, with 2 or 3 atoms being especially preferred.

When R¹ is Y, R² is preferably —CO₂R and R³ is R⁴ wherein R⁵ is Hal or CH₃. More preferably, when R¹ is Y, R² is preferably —CO₂R where R is CH₃, and R³ is R⁴ wherein R⁵ is F, Cl or I, most preferably I.

When R² is Y, R¹ is preferably C₁₋₄ fluoroalkyl, and R³ is R⁴ wherein R⁵ is Hal or CH₃. More preferably, when R² is Y, R¹ is preferably 3-fluoropropyl, and R³ is R⁴ wherein R⁵ is F or I, most preferably I.

In Formula (I), R³ is preferably Y. Preferred radioiodinated tropanes of the first aspect are thus of Formula (III):

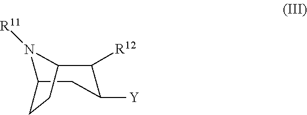

where R¹¹ is C₁₋₄ fluoroalkyl; and
R¹² is —CO₂R, where R is as defined in Formula (I).

In Formula (III), it is preferred that R¹¹ is 3-fluoropropyl and R¹² is —CO₂CH₃. More preferably, R¹¹ is 3-fluoropropyl and R¹² is —CO₂CH₃ and the linker group (L¹) is either an alkylene chain —(CH₂)ₙ— or —(C₆H₄)-4-(CH₂)ₙ— where each n is independently an integer of value 0 to 4, preferably 0 or 1, more preferably 0. The linker group in Formula (III) is thus preferably either absent or a para-phenylene linker. It is most preferably absent. These preferred embodiments of Formula III are illustrated in Schemes 1 to 3 of the second aspect (below).

In Formula (I), L¹ is preferably absent. The radioiodinated tropanes of the first aspect can be obtained by the method of preparation of the second and third aspects (below).

In a second aspect, the present invention provides a method of preparation of the radioiodinated tropane of Formula (I) as defined in the first aspect, where said method comprises:
(i) provision of a precursor of Formula (IA)

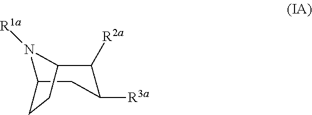

where:
R^{1a} is C₁₋₄ alkyl, C₁₋₄ fluoroalkyl or Y^{a};
R^{2a} is —CO₂R or Y^{a}, wherein R is C₁₋₄ alkyl, C₅₋₈ aryl or C₅₋₁₀ aralkyl;
R^{3a} is Y^{a} or R^{4a}, where R^{4a} is of formula:

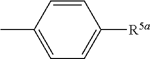

where R^{5a} is Hal, CH₃ or Y^{a};
Y^{a} is a Y^{1a} or Y^{2a} group:

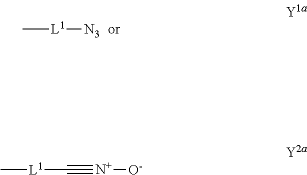

L¹ is a linker group which may be present or absent;
wherein R^{1a} to R^{5a} are chosen such that the precursor of Formula (IA) comprises one Y^{a} group;
(ii) reaction of said precursor with a compound of Formula (II):

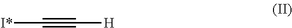

in the presence of a click cycloaddition catalyst, to give the radioiodinated tropane of Formula (I) via click cycloaddition,
wherein I* is a radioisotope of iodine, as defined in the first aspect.

In the second aspect, the groups R, L¹ and I* including preferred aspects thereof are as defined in the first aspect (above).

By the term “click cycloaddition catalyst” is meant a catalyst known to catalyse the click (alkyne plus azide) or click (alkyne plus isonitrile oxide) cycloaddition reaction of the first aspect. Suitable such catalysts are known in the art for use in click cycloaddition reactions. Preferred such catalysts include Cu(I), and are described below. Further details of suitable catalysts are described by Wu and Fokin [Aldrichim.Acta, 40(1), 7-17 (2007)] and Meldal and Tornoe [Chem. Rev., 108, 2952-3015 (2008)].

The click radioiodination method of the second aspect may be effected in a suitable solvent, for example acetonitrile, a C₁₋₄ alkylalcohol, dimethylformamide, tetrahydrofuran, or dimethylsulfoxide, or aqueous mixtures of any thereof, or in water. Aqueous buffers can be used in the pH range of 4-8, more preferably 5-7. The reaction temperature is preferably 5 to 100° C., more preferably at 75 to 85° C., most preferably at ambient temperature (typically 15-37° C.). The click cycloaddition may optionally be carried out in the presence of an organic base, as described by Meldal and Tornoe [Chem. Rev. 108, (2008) 2952, Table 1 (2008)].

A preferred click cycloaddition catalyst comprises Cu(I). The Cu(I) catalyst is present in an amount sufficient for the reaction to progress, typically either in a catalytic amount or in excess, such as 0.02 to 1.5 molar equivalents relative to the compound of Formula (IIa) or (IIb). Suitable Cu(I) catalysts include Cu(I) salts such as CuI or [Cu(NCCH₃)₄][PF₆], but advantageously Cu(II) salts such as copper (II) sulfate may be used in the presence of a reducing agent to generate Cu(I) in situ. Suitable reducing agents include: ascorbic acid or a salt thereof for example sodium ascorbate, hydroquinone, metallic copper, glutathione, cysteine, Fe²⁺, or Co²⁺. Cu(I) is also intrinsically present on the surface of elemental copper particles, thus elemental copper, for example in the form of powder or granules may also be used as catalyst. Elemental copper, with a controlled particle size is a preferred source of the Cu(I) catalyst. A more preferred such catalyst is elemental copper as copper powder, having a particle size in the range 0.001 to 1 mm, preferably 0.1 mm to 0.7 mm, more preferably around 0.4 mm. Alternatively, coiled copper wire can be used with a diameter in the range of 0.01 to 1.0 mm, preferably 0.05 to 0.5 mm, and more preferably with a diameter of 0.1 mm. The Cu(I) catalyst may optionally be used in the presence of bathophenanthroline, which is used to stabilise Cu(I) in click chemistry.

In Formula (IA), when R^{1a} is Y^{a}, R^{2a} is preferably —CO₂R and R^{3a} is R^{4a} wherein R^{5a} is Hal or CH₃. More preferably, when R^{1a} is Y^{a}, R^{2a} is preferably —CO₂R where R is CH₃, and R^{3a} is R^{4a} wherein R^{5a} is F, Cl or I, most preferably I.

In Formula (IA), when R^{2a} is Y^{a}, R^{1a} is preferably C₁₋₄ fluoroalkyl, and R^{3a} is R^{4a} wherein R^{5a} is Hal or CH₃. More preferably, when R^{2a} is Y^{a}, R^{1a} is preferably 3-fluoropropyl, and R^{3a} is R^{4a} wherein R^{5a} is F or I, most preferably I.

In Formula (IA), R^{3a} is preferably Y^{a}. When R^{3a} is Y^{a}, R^{1a} is preferably C₁₋₄ fluoroalkyl, and R^{2a} is —CO₂R. More preferably, when R^{3a} is Y^{a}, R^{1a} is preferably 3-fluoropropyl, and R^{2a} is —CO₂CH₃. Most preferably, when R^{3a} is Y^{a}, R^{1a} is preferably 3-fluoropropyl, R^{2a} is —CO₂CH₃ and the linker group (L¹) is an alkylene chain —(CH₂)ₙ— where n is an integer of value 0 to 4, preferably 0 or 1, more preferably 0. These preferred embodiments are illustrated in Schemes 1 to 4:

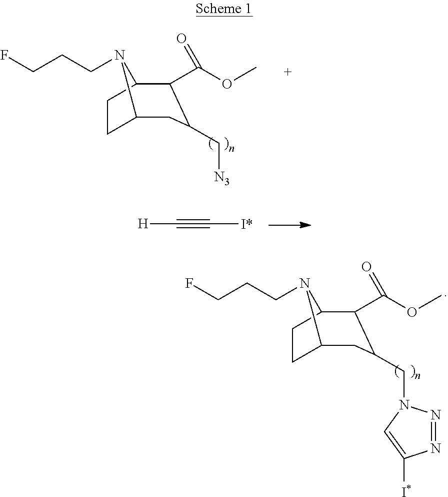

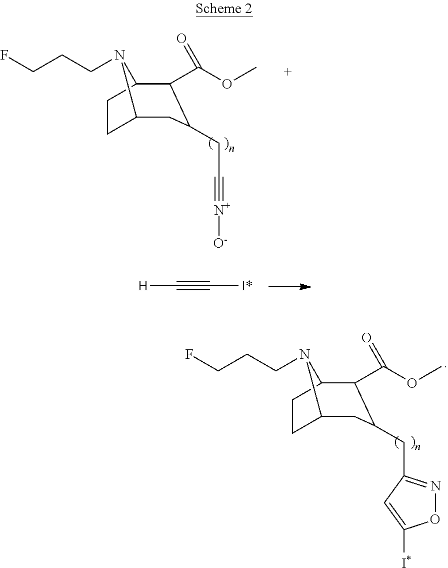

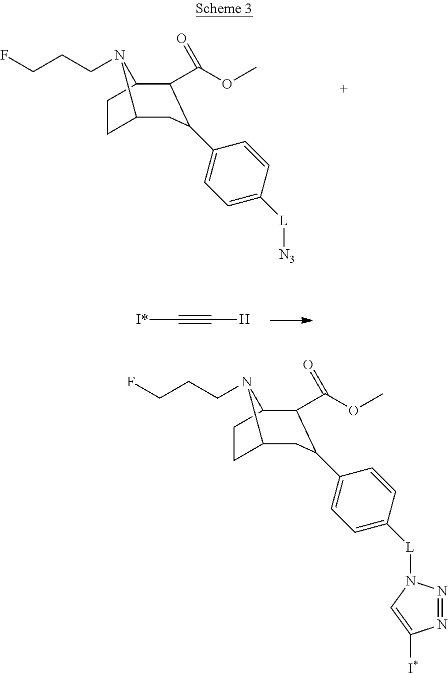

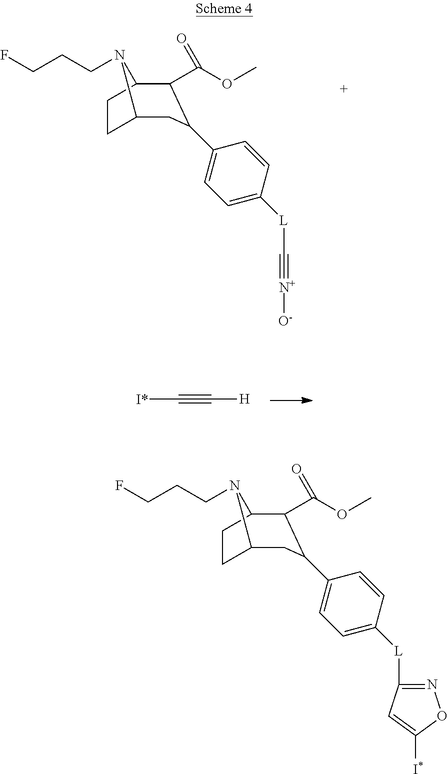

In Schemes 1 and 2, n is an integer of value 0 to 4, preferably 0 or 1, most preferably 0. In Schemes 3 and 4, L¹ is -(1,4-phenylene)-L- where L is -(A)ₘ₋₁- where A is as defined above. L is preferably —(CH₂)ₙ—. The synthesis of ¹²³I-iodoacetylene is described in Example 1.

In the method of the second aspect, the compound of Formula (II) may preferably be generated in situ by deprotection of a compound of Formula (IIa):

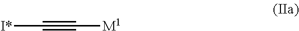

wherein M¹ is an alkyne-protecting group, and I* is as defined for Formula (I). Preferred aspects of I* in Formula (IIa), are as described for Formula (I).

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Suitable alkyne protecting groups are described in ‘Protective Groups in Organic Synthesis’, Theodora W. Greene and Peter G. M. Wuts, Chapter 8, pages 927-933, 4^{th} edition (John Wiley & Sons, 2007), and include: an trialkylsilyl group where each alkyl group is independently C₁₋₄ alkyl; an aryldialkylsilyl group where the aryl group is preferably benzyl or biphenyl and the alkyl groups are each independently C₁₋₄ alkyl; hydroxymethyl or 2-(2-hydroxypropyl). A preferred such alkyne protecting group is trimethylsilyl. The protected iodoalkynes of Formula IIa have the advantages that the volatility of the radioactive iodoalkyne can be controlled, and that the desired alkyne of Formula (II) can be generated in a controlled manner in situ, so that the efficiency of the reaction with the precursor of Formula (IA) is maximised.

The non-radioactive precursor of Formula (IA) may be prepared by the methods of: Carroll et al [J. Med. Chem., 35, 1813-1817 (1992)]; Lever et al, [Nucl. Med. Biol., 23, 277-284 (1996) and Bioconj. Chem., 16, 644-649 (2005]; Zou et al [J. Med. Chem., 44, 4453-4461 (2001)]; Vaughan et al [J. Neurosci., 19(2), 630-636 (1999)] and Nielsen et al [Bioorg. Med. Chem., 17, 4900-4909 (2009)]. General methods for the synthesis of azides are described in March's Advanced Organic Chemistry, fifth edition, M. B. Smith and John Wiley & Sons 2001), pages 1658 which summarises azide synthetic methods and the associated book sections.

The nitrile oxides of Formula (IA) where Y^{a} is Y^{2a}, can be obtained by the methods described by Ku et al [Org. Lett., 3(26), 4185-4187 (2001)], and references therein. Thus, they are typically generated in situ by treatment of an alpha-halo aldoxime with an organic base such as triethylamine. A preferred method of generation, as well as conditions for the subsequent click cyclisation to the desired isoxazole are described by Hansen et al [J. Org. Chem., 70(19), 7761-7764 (2005)]. Hansen et at generate the desired alpha-halo aldoxime in situ by reaction of the corresponding aldehyde with chloramine-T trihydrate, and then dechlorinating this with sodium hydroxide. The corresponding aldoxime is prepared by reacting the corresponding aldehyde with hydroxylamine hydrochloride at pH 9-10. See also K. B. G. Torsell Nitrite Oxides, Nitrones and Nitronates in Organic Synthesis [VCH, New York (1988)].

The radioiodinated alkyne of Formula (II) can be obtained as follows:

(i) reaction of a precursor of either Formula IV or Formula V

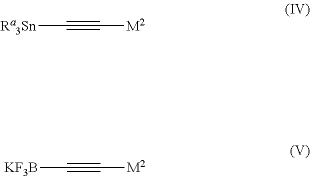

wherein M² is H or an M¹ group, and M¹ is as defined in the second aspect, and each R^{a} is independently C₁₋₄ alkyl;
with a supply of radioactive iodide ion in the presence of an oxidising agent, to give a compound of Formula IIb:

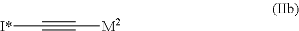

where I* is as defined in the first aspect;
   (ii) when M² is an M¹ group, deprotection to remove the M¹ group.

Suitable protecting groups M¹ are as described above. Deprotection conditions are described in Protective Groups in Organic Synthesis, Theodora W. Greene and Peter G. M. Wuts, Chapter 8, pages 927-933, 4^{th} edition (John Wiley & Sons, 2007).

The precursor of Formula IV or V is non-radioactive. Some precursors of Formula (IV) are commercially available. Thus, the trialkyltin compounds Bu₃Sn—≡—H and Bu₃Sn—≡—SiMe₃ are commercially available from Sigma-Aldrich. Other organotin intermediates are described by Ali et al [Synthesis, 423-445 (1996)]. Suitable oxidising agents are described by Bolton [J. Lab. Comp. Radiopharm., 45, 485-528 (2002)]. Preferred oxidising agents are peracetic acid (which is commercially available) at pH ca. 4, and hydrogen peroxide/aqueous HCl at pH ca. 1. When M² is H, the compound of Formula IIb is iodoacetylene. The synthesis of the non-radioactive (¹²⁷I) analogue has been described by Ku et al [Org. Lett., 3(26), 4185-4187 (2001)]. The synthesis of ¹²³I-labelled alkynyl iodides via the potassium alkynyltrifluoroborate precursors analogous to Formula (V), using peracetic acid in the radioiodination step, has been described by Kabalka et al [J. Lab. Comp. Radiopharm., 48, 359-362 (2005)]. The synthesis of potassium alkynyltrifluoroborate precursors from the corresponding alkyne is described therein, as well as in Kabalka et al [J. Lab. Comp. Radiopharm., 49, 11-15 (2006)]. The potassium alkynyltrifluoroborate precursors are stated to be crystalline solids, which are stable to both air and water.

In a third aspect, the present invention provides a method of preparation of the radioiodinated tropane of Formula (I) as defined in the first aspect, where said method comprises:
(i) provision of a precursor of Formula (IB):

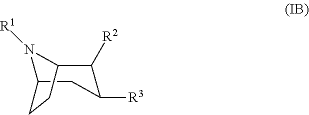

where:
R¹ is C₁₋₄ alkyl, C₁₋₄ fluoroalkyl or Y^{b};
R² is —CO₂R or Y^{b}, wherein R is C₁₋₄ alkyl, C₅₋₈ aryl or C₅₋₁₀ aralkyl;
R³ is Y^{b} or R⁴, where R⁴ is of formula:

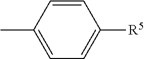

where R⁵ is Hal, CH₃ or Y^{b};
Y^{b} is a Y^{2b} group:

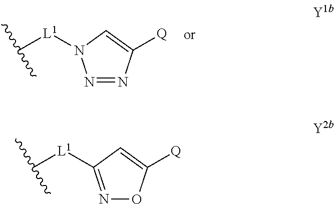

L¹ is a linker group which may be present or absent; wherein Q is R^{a}₃Sn— or KF₃B-, where each R^{a} is independently C₁₋₄ alkyl; and wherein R¹ to R⁵ are chosen such that the precursor of Formula (IB) comprises one Y^{b} group;
(ii) reaction of said precursor with radioactive iodide ion in the presence of an oxidising agent to give the radioiodinated tropane of Formula (I).

In the third aspect, the groups R, L¹ and I* including preferred aspects thereof are as defined in the first aspect (above). Q is preferably R^{a}₃Sn—. Y^{b} is preferably Y^{1b}.

By the term “oxidising agent” is meant an oxidant capable of oxidising iodide ion to form the electrophilic species (HOI, H₂OI), wherein the active iodinating agent is I⁺. Suitable oxidising agents are described by Bolton [J. Lab. Comp. Radiopharm., 45, 485-528 (2002)], and Eersels et al [J. Lab. Comp. Radiopharm., 48, 241-257 (2005)] and include peracetic acid and N-chloro compounds, such as chloramine-T, iodogen, iodogen tubes and succinimides. Preferred oxidising agents are peracetic acid (which is commercially available) at pH ca. 4, and hydrogen peroxide/aqueous HCl at pH ca. 1. Iodogen tubes are commercially available from Thermo Scientific Pierce Protein Research Products.

By the term “radioactive iodide ion” is meant a radioisotope of iodine (as defined above), in the chemical form of iodide ion (I⁻).

When Q is R^{a}₃Sn—, the radioiodination method of the third aspect is carried out as described by Bolton [J. Lab. Comp. Radiopharm., 45, 485-528 (2002)] and Eersels et al [J. Lab. Comp. Radiopharm., 48, 241-257 (2005)]. The organotin precursors are prepared as described by Ali et al [Synthesis, 423-445 (1996)].

When Q is KF₃B-, the radioiodination reaction method of the third aspect can be carried out as described by Kabalka et al [J. Lab. Comp. Radiopharm., 48, 359-362 (2005)], who use peracetic acid as the oxidising agent. Precursors where Q is KF₃B- can be obtained from the corresponding alkyne as described by Kabalka et al [J. Lab. Comp. Radiopharm., 48, 359-362 (2005) and, J. Lab. Comp. Radiopharm., 49, 11-15 (2006)]. The potassium trifluoroborate precursors are stated to be crystalline solids, which are stable to both air and water.

The radioiodination reaction of the third aspect may be effected in a suitable solvent, for example acetonitrile, a C₁₋₄ alkylalcohol, dimethylformamide, tetrahydrofuran (THF), or dimethylsulfoxide, or mixtures thereof, or aqueous mixtures thereof, or in water. Aqueous buffers can also be used. The pH will depend on the oxidant used, and will typically be pH 0 to 1 when eg. hydrogen peroxide/aqueous acid is used, or in the range pH 6-8 when iodogen or iodogen tubes are used. The radioiodination reaction temperature is preferably 10 to 60° C., more preferably at 15 to 50° C., most preferably at ambient temperature (typically 15-37° C.). Organic solvents such as acetonitrile or THF and/or the use of more elevated temperature may conveniently be used to solubilise any precursors of Formula (IB) which are poorly soluble in water.

The method of preparation of the second or third aspect is preferably carried out in an aseptic manner, such that the product of Formula (I) is obtained as a radiopharmaceutical composition. Further description of radiopharmaceutical composition is given in the fourth aspect (below). Thus, the method is carried out under aseptic manufacture conditions to give the desired sterile, non-pyrogenic radiopharmaceutical product. It is preferred therefore that the key components, especially any parts of the apparatus which come into contact with the product of Formula (I) (e.g. vials and transfer tubing) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise the non-radioactive components in advance, so that the minimum number of manipulations need to be carried out on the radioiodinated radiopharmaceutical product. As a precaution, however, it is preferred to include at least a final sterile filtration step.

The precursor of Formula (IA) or (IB), plus other reagents and solvents are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour. The reaction vessel is suitably chosen from such containers, and preferred embodiments thereof. The reaction vessel is preferably made of a biocompatible plastic (eg. PEEK).

The method of the second or third aspect is preferably carried out using an automated synthesizer apparatus. By the term “automated synthesizer” is meant an automated module based on the principle of unit operations as described by Satyamurthy et al [Clin. Positr. Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical product is desired. They are commercially available from a range of suppliers [Satyamurthy et al, above], including: GE Healthcare; CTI Inc; Ion Beam Applications S. A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours. The automated synthesizer preferably comprises a cassette. By the term “cassette” is meant a piece of apparatus designed to fit removably and interchangeably onto an automated synthesizer apparatus (as defined below), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (eg. SPE). The cassette always comprises a reaction vessel. Such reaction vessels are preferably 1 to 10 cm³, most preferably 2 to 5 cm³ in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radio lysis.

Preferred automated synthesizers of the present invention are those which comprise a disposable or single use cassette which comprises all the reagents, reaction vessels and apparatus necessary to carry out the preparation of a given batch of radioiodinated radiopharmaceutical. The cassette means that the automated synthesizer has the flexibility to be capable of making a variety of different radioiodine-labelled radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

In a fourth aspect, the present invention provides a radiopharmaceutical composition comprising an effective amount of a compound of Formula (I) according to the first aspect, together with a biocompatible carrier medium. Preferred embodiments of the radioiodinated tropane of Formula (I) in the fourth aspect are as described in the first aspect (above).

The “biocompatible carrier medium” comprises one or more pharmaceutically acceptable adjuvants, excipients or diluents. It is preferably a fluid, especially a liquid, in which the compound of Formula (I) is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

In a fifth aspect, the present invention provides the use of the precursor of Formula (IA) as defined in the second aspect, or the precursor of Formula (IB) as defined in the third aspect for the manufacture of the radioiodinated tropane of Formula (I) as defined in the first aspect, or for the manufacture of the radiopharmaceutical composition of the fourth aspect.

Preferred aspects of the precursors, radioiodinated tropane and radiopharmaceutical composition in the fifth aspect are as described above.

In a sixth aspect, the present invention provides the use of an automated synthesizer apparatus to carry out the method of the second or third aspect.

The automated synthesizer apparatus and preferred embodiments thereof are as described in the second and third aspects (above).

In a seventh aspect, the present invention provides method of generating an image of a human or animal body comprising administering a radioiodinated tropane according to the first aspect, or the radiopharmaceutical composition according to the fourth aspect and generating an image of at least a part of said body to which said tropane or composition has distributed using PET or SPECT. The image is expected to be useful in the imaging of the dopamine transporter in vivo, and hence in particular Parkinsonian syndromes, including Parkinson's disease; DLB (Lewy Body Dementia) and AD-HD (Attention Deficit Hyperactivity Disorder).

In a further aspect, the present invention provides a method of monitoring the effect of treatment of a human or animal body with a drug, said method comprising administering to said body a radioiodinated tropane according to the first aspect, or the composition according to the fourth aspect, and detecting the uptake of said compound or composition in at least a part of said body to which said compound or composition has distributed using PET or SPECT.

The administration and detection of this final aspect are preferably effected before and after treatment with said drug, so that the effect of the drug treatment on the human or animal patient can be determined. Where the drug treatment involves a course of therapy, the imaging can also be carried out during the treatment.

The invention is illustrated by the following Examples. Example 1 provides the synthesis of ¹²³I-iodoacetylene. Example 2 provides the click cycloaddition of ¹²³I-iodoacetylene to an azide derivative, to form a radioiodinated triazole ring. Example 3 provides the click cycloaddition of ¹²³I-iodoacetylene to an isonitrile oxide derivative, to form a radioiodinated isoxazole ring. Example 4 provides a click cycloaddition of a tributyltin-alkyne to an azide derivative, to form a triazole radioiodination precursor having a triazole-tributyltin bond. Example 5 provides the conditions for converting the precursor of Example 4, to the radioiodinated product. Example 6 provides a synthesis of an isoxazole radioiodination precursor having an isoxazole-tributyltin bond via click cycloaddition from an isonitrile oxide derivative.

Example 7 provides the synthesis of a radioiodinated isoxazole via the precursor of Example 6. Example 8 provides the synthesis of an azide-functionalised tropane. Example 9 provides the synthesis of a (tributyltin)triazole-functionalised tropane. Example 10 provides the synthesis of an aldehyde-functionalised tropane. Example 11 provides the synthesis of a (tributyltin)isoxazole-functionalised tropane. Example 12 provides the synthesis of a radioiodinated triazole-functionalised tropane. Example 13 provides the synthesis of a radioiodinated isoxazole-functionalised tropane.

Abbreviations.

DMF: Dimethylformamide,

HPLC: High performance liquid chromatography,

MeCN: Acetonitrile,

PAA: Peracetic acid,

RCP: radiochemical purity,

RT: room temperature.

### EXAMPLE 1

### Preparation and Distillation of [¹²³I]-Iodoacetylene Using Peracetic Acid Oxidant

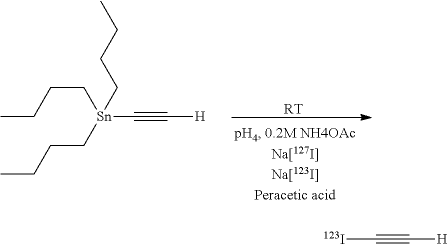

To a Wheaton vial on ice was added, ammonium acetate buffer (100 μl, 0.2M, pH 4), sodium [¹²⁷I] iodide (10 μl, 10 mM solution in 0.01M sodium hydroxide, 1×10⁻⁷ moles), sodium [¹²³I] iodide (20 μl, 53 MBq), peracetic acid, (10 μl, 10 mM solution, 1×10⁻⁷ moles) and a solution of ethynyltributylstannane in THF (Sigma-Aldrich; 38 μl, 1 mg/ml, 1.2×10⁻⁷ moles). Finally, 460 μl THF was added, the Wheaton vial sealed and the reaction mixture allowed to warm to room temperature prior to reverse phase HPLC analysis which showed [¹²³I]-iodoacetylene with a radiochemical purity (RCP) of 75% (t_{R} 12.3 minutes, System A).

The reaction mixture was heated at 80-100° C. for 30 minutes during which time, the [¹²³I]-iodoacetylene and THF were distilled through a short tube into a collection vial on ice. After this time, a low flow of nitrogen was passed through the septa of the heated vial to remove any residual liquids from the tube. [¹²³I]-iodoacetylene was collected in 38.6% yield (non decay corrected) with an RCP of 94%. (t_{R} 12.3 minutes, System A).

HPLC System A

A=water

B=acetonitrile

Column C18 (2) phenonenex Luna, 150×4.6 mm, 5 micron

| | Time (min) | 0 | 1 | 20 | 25 | 25.5 | 30 |
|---|---|---|---|---|---|---|---|
| Gradient | % B | 5 | 5 | 95 | 95 | 5 | 5 |

### EXAMPLE 2

### Preparation of 1-Benzene-4-[¹²³I]-iodo-1H-1,2,3-triazole (Prophetic Example)

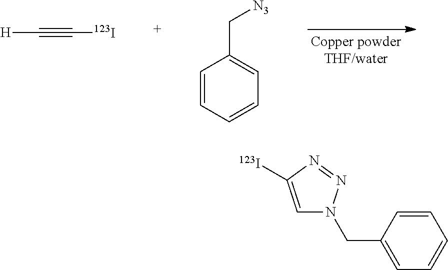

To a Wheaton vial charged with copper powder (200 mg, −40 mesh), sodium phosphate buffer (200 μL, pH 6, 50 mM) and placed on ice is added, [¹²³I]-iodoacetylene and benzyl azide (1 mg, 7.5×10⁻⁶ moles). Following reagent addition, the ice bath is removed and the reaction incubated at room temperature with heating applied as required. 1-Benzene-4-[¹²³I]-iodo-1H-1,2,3-triazole is purified by reverse phase HPLC.

### EXAMPLE 3

### Preparation of 5-[¹²³I]-Iodo-3-phenyl isoxazole (Prophetic Example)

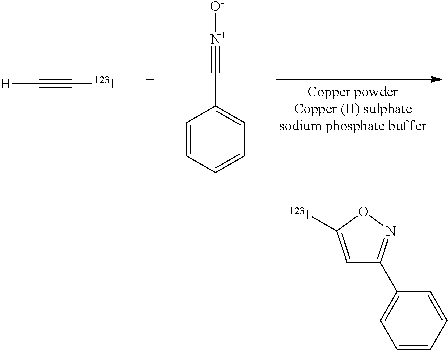

To a Wheaton vial charged with copper powder (50 mg, −40 mesh), copper (II) sulfate (3.8 μg, 1.53×10−8 moles, 0.5 mg/mL solution in water), sodium phosphate buffer (100 μL, 50 mM, pH 6) and placed on ice, is added [¹²³I]-iodoacetylene and benzonitrile-N-oxide (1 mg, 8.4×10⁻⁶ moles. Following reagent addition, the ice bath is removed and the reaction incubated at room temperature with heating applied as required. 5-[¹²³I]-iodo-3-phenyl isoxazole is purified by reverse phase HPLC.

### EXAMPLE 4

### Preparation of 1-Phenyl-4-(tributylstannyl)-1H [1,2,3] triazole (Prophetic Example)

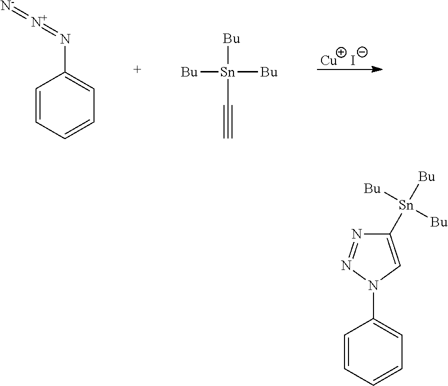

Phenylazide can be obtained from Sigma-Aldrich or can be synthesized by the method described in J. Biochem., 179, 397-405 (1979). A solution of tributylethynylstannane (Sigma Aldrich; 400 mg, 1.27 mmol) in THF (4 ml) is treated with phenylazide (169 mg, 1.27 mmol), copper (I) iodide (90 mg, 0.47 mmol), and triethylamine (256 mg, 2.54 mmol) at room temperature over 48 h. The reaction is then filtered through celite to remove copper (I) iodide and chromatographed on silica in a gradient of 5-20% ethyl acetate in petrol. The second fraction is collected and concentrated in vacuo to give the 1-phenyl-4-(tributylstannyl)-1H [1,2,3] triazole as a colourless oil.

### EXAMPLE 5

### Preparation of [¹²³I]-1-phenyl-4-iodo-1H [1,2,3] triazole Using Peracetic Acid as the Oxidant (Prophetic Example)

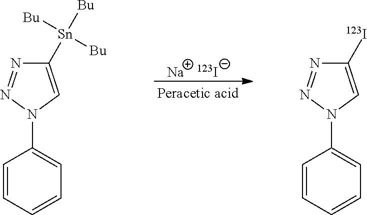

To sodium [¹²³I] iodide, received in 5-20 μL 0.05M sodium hydroxide is added ammonium acetate buffer (100 μL pH 4.0, 0.2M), sodium [¹²⁷I] iodide (10 μL 1 mM solution in 0.01M sodium hydroxide, 1×10⁻⁸ moles), peracetic acid (PAA) solution (10 μL 1 mM solution, 1×10⁻⁸ moles) and finally, 1 phenyl-4-tributylstannyl-1H [1,2,3] triazole (Example 4; 43 μg, 1×10⁻⁷ moles) dissolved in acetonitrile. The reaction mixture is incubated at room temperature for 15 minutes prior to purification by HPLC.

### EXAMPLE 6

### Preparation of 3-Phenyl-5-(tributylstannyl)isoxazole (Prophetic Example)

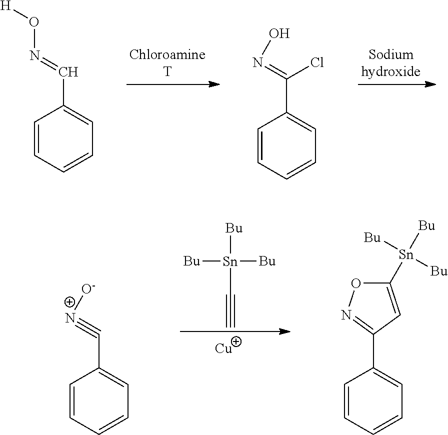

(E)-benzaldehyde oxime (Sigma Aldrich; 3.3 g, 20 mmol) in tent butanol and water (1:1) 80 ml, is treated with chloramine T trihydrate (Sigma Aldrich; 5.9 g, 21 mmol) in small, portions over 5 min. The reaction is then treated with copper sulfate pentahydrate (0.15 g, 0.6 mmol) and copper turnings ˜50 mg and tributylethynylstannane (6.3 g, 20 mmol). The reaction is then adjusted to pH 6 with sodium hydroxide solution and stirred for 6 h. The reaction mixture is treated with dilute ammonium hydroxide solution to remove all copper salts. The product is collected by filtration, redissolved in ethyl acetate and filtered through a short plug of silica gel. The filtrate is concentrated in vacuo to give 3-phenyl-5-(tributylstannyl) isoxazole.

### EXAMPLE 7

### Preparation of 5-[¹²³I]-Iodo-3-phenyl isoxazole (Prophetic Example)

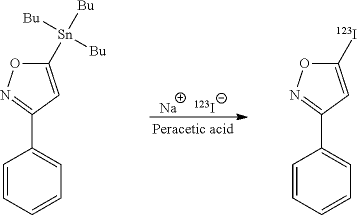

To sodium [¹²³I] iodide, received in 5-20 μL 0.05M sodium hydroxide is added ammonium acetate buffer (100 μL pH 4.0, 0.2M), sodium [¹²⁷I] iodide (10 μL, 1 mM solution in 0.01M sodium hydroxide, 1×10⁻⁸ moles), peracetic acid (PAA) solution (10 μL 1 mM solution, 1×10⁻⁸ moles) and finally, 3-phenyl-5-tributylstannyl-isoxazole (Example 6; 43 μg, 1×10⁻⁷ moles) dissolved in acetonitrile. The reaction mixture is incubated at room temperature for 15 minutes prior to purification by HPLC.

### EXAMPLE 8

### Preparation of (1R,2R,5S)-Methyl 3-azido-8-(3-fluoropropyl)-8-azabicyclo[3,2,1]octane-2-carboxylate (Prophetic Example)

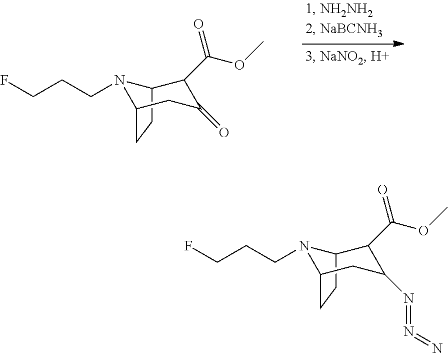

The conversion to the azide uses a method similar to that described in Tetrahedron Letters; vol. 41(49); p. 9575-9580 (2000). Thus, (1R,2R,5S) methyl 8-(3-fluoropropyl)-3-oxo-8-azabicyclo[3,2,1]octane [1 g, 4.1 mmol; prepared as described in Tetrahedron Letters Vol 37(31), 5479-5482 (1996)] dissolved in methanol (10 ml) is treated with hydrazine (131 mg 4.1 mmol), and allowed to stand at room temperature for 2 h. The reaction mixture is then treated with sodium cyanoborohydride (516 mg, 8.2 mmol), and adjusted to pH 4 with 1N hydrochloric acid. The reaction is allowed to stand at room temperature for 3 h, and then treated with sodium nitrite (276 mg, and the reaction allowed to stand at room temperature for a further 2 h. The reaction is then concentrated in vacuo to a gum, and partitioned between ethyl acetate and sodium bicarbonate solution. The ethyl acetate solution was then concentrated in vacuo to a gum and chromatographed on silica in a gradient of 5-20% ethyl acetate in petrol to give (1R,2R,5S)-Methyl 3-azido-8-(3-fluoropropyl)-8-azabicyclo[3,2,1]octane-2-carboxylate.

### EXAMPLE 9

### Preparation of (1R,2R,5S)-Methyl 3-(4tributylstannyl)1H-1,2,3,triazole-1yl)-8-(3-fluoropropyl)-8-azabicyclo[3,2,1]octane-2-carboxylate (Prophetic Example)

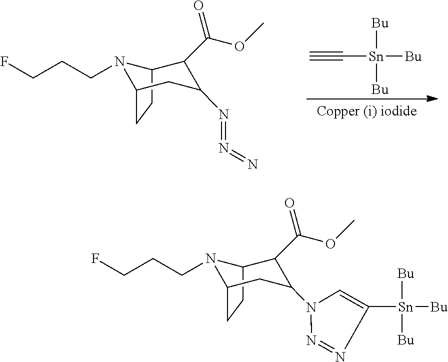

(1R,2R,5S)-Methyl 3-azido-8-(3-fluoropropyl)-8-azabicyclo[3,2,1]octane-2-carboxylate (1 g, 3.7 mmol) in THF (50 ml) is treated with trimethylethynylstannane (703 mg 3.7 mmol) and copper (I) iodide (50 mg), and the reaction then heated under reflux for 2 h. The reaction mixture is then allowed to cool, and then concentrated in vacuo to give a gum and this is purified by chromatography on silica in a gradient of 5-50% ethyl acetate in petrol to give (1R,2R,5S)-Methyl 3-(4-tributylstannyl)1H-1,2,3,triazole-1yl)-8-(3-fluoropropyl)-8-azabicyclo[3,2,1]octane-2-carboxylate.

### EXAMPLE 10

### Preparation of 1R,2S,5S,)-methyl 8-(3-fluoropropyl)-3-formyl-8-azabicyclo[3.2.1]octane-2-carboxylate (Prophetic Example)

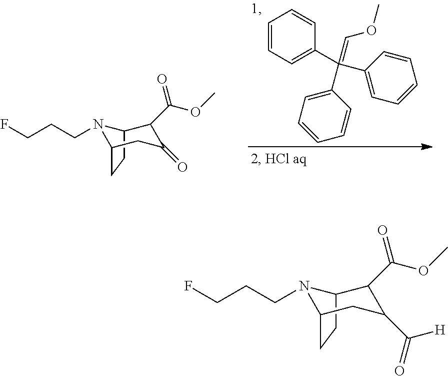

To (1R,2R,5S) methyl 8-(3-fluoropropyl)-3-oxo-8-azabicyclo[3,2,1]octane (1 g, 4.1 mmol) prepared as described in Example 8 in THF (50 ml) is reacted with (methoxymethyl)triphenylphosphorane (4.1 mmol; prepared from the corresponding ylid by deprotonation with sodium hydride to give the vinyl ether). The vinyl ether is hydrolysed directly by the addition of 1N hydrochloric acid and heating at reflux for 2 h. The reaction is concentrated in vacuo to remove most of the THF, and the product recovered by partition between water and ethyl acetate. The ethyl acetate solution is dried over sodium sulfate and concentrated in vacuo to give a gum that is purified by chromatography on silica in a gradient of 10-30% ethyl acetate in petrol to give (1R,2S,5S,)-methyl 8-(3-fluoropropyl)-3-formyl-8-azabicyclo[3.2.1]octane-2-carboxylate as the main fraction.

### EXAMPLE 11

### Preparation of (1R,2S,5S,)-methyl 8-(3-fluoropropyl)-3-(5-(tributylstannyl)oxazol-2yl)-8-azabicyclo[3.2.1]octane-2-carboxylate (Prophetic Example)

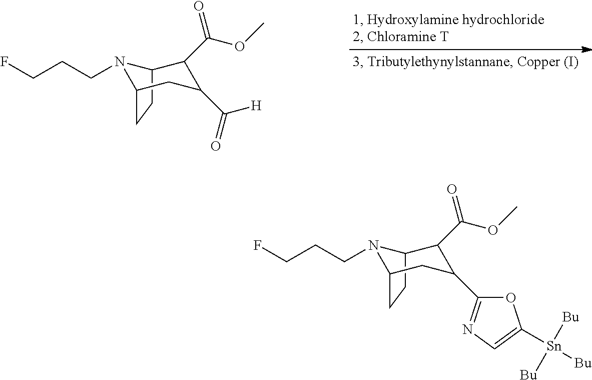

(1R,2S,5S,)-methyl 8-(3-fluoropropyl)-3-formyl-8-azabicyclo[3.2.1]octane-2-carboxylate (1 g, 3.8 mmol) in acetonitrile (50 ml) is reacted with hydroxylamine hydrochloride (270 mg, 3.8 mmol) and sodium hydroxide (152 mg, 3.8 mmol), and the reaction mixture then stirred at room temperature for 2 h. To this mixture is added chloramine T (3.8 mmol) and the reaction stirred at room temperature for 15 minutes. A further portion of sodium hydroxide (152 mg, 3.8 mmol) is then added, and the reaction stirred for a further 15 minutes. The reaction mixture is then treated with tributylethynylstannane (1.187 g, 3.8 mmol) and copper (I) chloride (50 mg). The reaction is then concentrated in vacuo and the product recovered by partitioning between ethyl acetate and water. The ethyl acetate layer is separated, dried over sodium sulfate and concentrated in vacuo to a gum. The gum is then chromatographed on silica in a gradient of 5-20% ethyl acetate in petrol. The main fraction was collected to give (1R,2S,5S,)-methyl 8-(3-fluoropropyl)-3-(5-(tributylstannyl)oxazol-2yl)-8-azabicyclo[3.2.1]octane-2-carboxylate.

### EXAMPLE 12

### Preparation of [¹²³I]-(1R,2R,5S)-Methyl 3-(Iodo)1H-1,2,3,triazole-1yl)-8-(3-fluoropropyl)-8-azabicyclo[3,2,1]octane-2-carboxylate (Prophetic Example)

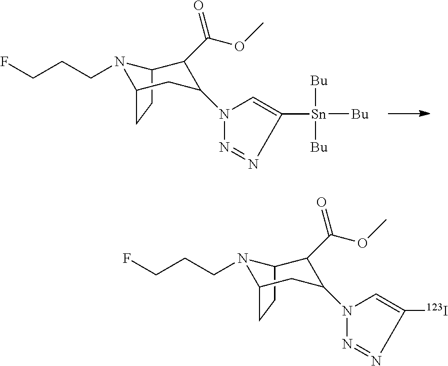

To sodium [¹²³I] iodide, received in 5-20 μL 0.05M sodium hydroxide is added ammonium acetate buffer (100 μL pH 4.0, 0.2M), sodium [¹²⁷I] iodide (10 μL, 1 mM solution in 0.01M sodium hydroxide, 1×10^{31 8} moles), peracetic acid (PAA) solution (10 μL 1 mM solution, 1×10⁻⁸ moles) and finally (1R,2R,5S)-Methyl 3-(4-tributylstannyl)1H-1,2,3,triazole-1yl)-8-(3-fluoropropyl)-8-azabicyclo[3,2,1]octane-2-carboxylate (58 μg, 1×10⁻⁷ moles) dissolved in acetonitrile. The reaction mixture is allowed to stand at room temperature for 15 minutes prior to HPLC purification of the iodinated product [¹²³I]-(1R,2R,5S)-Methyl 3-(Iodo)1H-1,2,3,triazole-1yl)-8-(3-fluoropropyl)-8-azabicyclo[3,2,1]octane-2-carboxylate.

### EXAMPLE 13

### Preparation of [¹²³I]-(1R,2S,5S,)-methyl 8-(3-fluoropropyl)-3-(5-iodooxazol-2yl)-8-azabicyclo[3.2.1]octane-2-carboxylate (Prophetic Example)

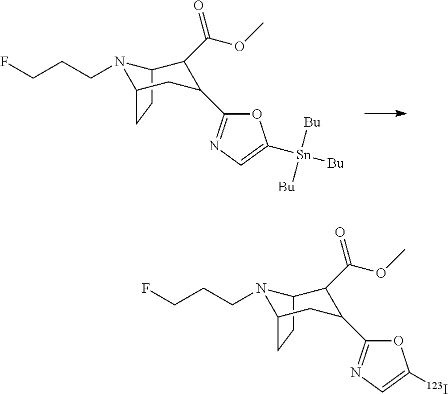

To sodium [¹²³I] iodide, received in 5-20 μL 0.05M sodium hydroxide is added ammonium acetate buffer (100 μL pH 4.0, 0.2M), sodium [¹²⁷I] iodide (10 μL, 1 mM solution in 0.01M sodium hydroxide, 1×10⁻⁸ moles), peracetic acid (PAA) solution (10 μL 1 mM solution, 1×10⁻⁸ moles) and finally (1R,2S,5S,)-methyl 8-(3-fluoropropyl)-3-(5-(tributylstannyl)oxazol-2yl)-8-azabicyclo[3.2.1]octane-2-carboxylate solution (58 μg, 1×10⁻⁷ moles) dissolved in acetonitrile. The reaction mixture is allowed to stand at room temperature for 15 minutes prior to HPLC purification of the iodinated product [¹²³I]-(1R,2S,5S,)-methyl 8-(3-fluoropropyl)-3-(5-iodooxazol-2yl)-8-azabicyclo[3.2.1]octane-2-carboxylate.

What is claimed is:

1. A radioiodinated tropane of Formula (I): 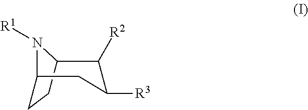
where:
R¹ is C₁₋₄ alkyl, C₁₋₄ fluoroalkyl or Y;
R² is —CO₂R or Y, wherein R is C₁₋₄ alkyl, C₅₋₈ aryl or C₅₋₁₀ aralkyl;
R³ is Y or R⁴, where R⁴ is of formula: 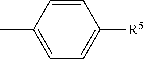
where R⁵ is Hal, CH₃ or Y;
Y is a Y¹ or Y² group: 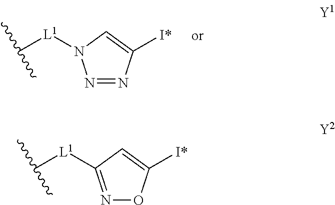
L¹ is a linker group which may be present or absent;
I* is a radioisotope of iodine;
wherein R¹ to R⁵ are chosen such that the tropane of Formula (I) comprises one Y group.

2. The radioiodinated tropane of claim 1, wherein I* is chosen from ¹²³I, ¹²⁴I or ¹³¹I.

3. The radioiodinated tropane of claim 1, where Y is Y¹.

4. The radioiodinated tropane of claim 1, where R¹ is Y, R² is —CO₂R and R³ is R⁴ wherein R⁵ is Hal or CH₃.

5. The radioiodinated tropane of claim 1, where R² is Y, R¹ is C₁₋₄ fluoroalkyl, and R³ is R⁴ wherein R⁵ is Hal or CH₃.

6. A method of preparation of the radioiodinated tropane of Formula (I) as defined in claim 1, where said method comprises:
(i) provision of a precursor of Formula (IA) 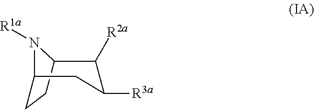
where:
R^{1a} is C₁₋₄ alkyl, C₁₋₄ fluoroalkyl or Y^{a};
R^{2a} is —CO₂R or Y^{a}, wherein R is C₁₋₄ alkyl, C₅₋₈ aryl or C₅₋₁₀ aralkyl;
R^{3a} is Y^{a} or R^{4a}, where R^{4a} is of formula: 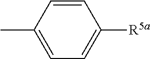
where R^{5a} is Hal, CH₃ or Y^{a};
Y^{a} is a Y^{1a} or Y^{2a} group: 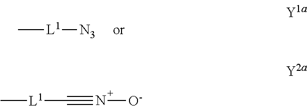
L¹ is a linker group which may be present or absent;
wherein R^{1a} to R^{5a} are chosen such that the precursor of Formula (IA) comprises one Y^{a} group;
(ii) reaction of said precursor with a compound of Formula (II): 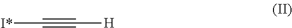
in the presence of a click cycloaddition catalyst, to give the radioiodinated tropane of Formula (I) via click cycloaddition,
wherein I* is a radioisotope of iodine, as defined in claim 1.

7. The method of claim 6, where the click cycloaddition catalyst comprises Cu(I).

8. The method of claim 6, where the compound of Formula (II) is generated in situ by deprotection of a compound of Formula (IIa): 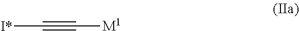
wherein M¹ is an alkyne-protecting group.

9. A method of preparation of the radioiodinated tropane of Formula (I) as defined in claim 1, where said method comprises:
(i) provision of a precursor of Formula (IB): 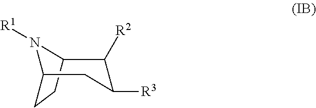
where:
R¹ is C₁₋₄ alkyl, C₁₋₄ fluoroalkyl or Y^{b};
R² is —CO₂R or Y^{b}, wherein R is C₁₋₄ alkyl, C₅₋₈ aryl or C₅₋₁₀ aralkyl;
R³ is Y^{b} or R⁴, where R⁴ is of formula: 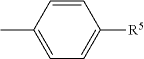
where R⁵ is Hal, CH₃ or Y^{b};
Y^{b} is a Y^{1b} or Y^{2b} group: 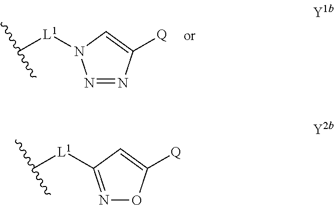
L¹ is a linker group which may be present or absent;
wherein Q is R^{a}₃Sn— or KF₃B—, where each R^{a} is independently C₁₋₄ alkyl; and wherein R¹ to R⁵ are chosen such that the precursor of Formula (IB) comprises one Y^{b} group;
(ii) reaction of said precursor with radioactive iodide ion in the presence of an oxidising agent to give the radioiodinated tropane of Formula (I).

10. The method of claim 6, which is carried out in an aseptic manner, such that the product of Formula (I) is obtained as a radiopharmaceutical composition.

11. The method of claim 6, which is carried out using an automated synthesizer apparatus.

12. A radiopharmaceutical composition comprising an effective amount of the radioiodinated tropane of Formula (I) as defined in claim 1, together with a biocompatible carrier medium.

13-14. (canceled)

15. A method of generating an image of a human or animal body comprising administering the radioiodinated tropane of Formula (I) as defined in the radiopharmaceutical composition of claim 12, and generating an image of at least a part of said body to which said compound or composition has distributed using PET or SPECT.

16. A method of monitoring the effect of treatment of a human or animal body with a drug, said method comprising administering to said body the radioiodinated tropane of Formula (I) as defined in the radiopharmaceutical composition of claim 12, and detecting the uptake of said tropane or composition in at least a part of said body to which said tropane or composition has distributed using PET or SPECT, said administration and detection optionally but preferably being effected before, during and after treatment with said drug.
